(19) United States
(12) Patent Application Publication
(10) Pub. No.: US 2006/0276495 A1
(43) Pub. Date: Dec. 7, 2006

(54) Treatment methods of nasal congestion and nasal obstruction

(75) Inventors: Luis M. Salmun, Short Hills, NJ (US); Patricia Rohane, Randolph, NJ (US); Richard R. Lorber, Scotch Plains, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 11/406,837

(22) Filed: Apr. 19, 2006

Related U.S. Application Data
(63) Continuation of application No. 10/177,366, filed on Jun. 20, 2002.
(60) Provisional application No. 60/299,636, filed on Jun. 20, 2001.
(60) Provisional application No. 60/299,637, filed on Jun. 20, 2001.

(51) Int. Cl. A61K 31/473

(52) U.S. Cl. 514/290

(57) ABSTRACT

The use of desloratadine and/or other antihistamines for treating and/or preventing severe nasal congestion and/or nasal blockage associated with allergic and inflammatory conditions of the upper and lower airway passages in a human.

This application claims formal benefit of priority to Provisional U.S. Patent Application Ser. Nos. 60/299,636 and 60/299,637, both of which were filed Jun. 20, 2001.

### FIELD OF THE INVENTION

This invention relates to the use of desloratadine and/or other antihistamines for treating and/or preventing nasal congestion and/or nasal blockage as defined by symptomatic complaints of nasal airflow limitation associated with allergic and inflammatory conditions, such as seasonal allergic rhinitis.

### BACKGROUND OF THE INVENTION

Desloratadine, disclosed in U.S. Pat. No. 4,659,716, is a non-sedating antihistamine useful for treating allergic reactions in animals including humans. U.S. Pat. No. 5,695,997 discloses pharmaceutical compositions containing desloratadine and methods of using desloratadine for treating and preventing disease states, e.g., allergic rhinitis.

Severe nasal congestion/stuffiness and/or blockage is a chronic symptom in patients with allergic disorders such as allergic rhinitis. However, currently available antihistamines have not been effective in treating severe nasal blockage and congestion/stuffiness associated with allergic disorders. Congestion associated with allergic rhinitis has been treated by the administration of combination products containing an antihistamine and the decongestant pseudoephedrine. For example, the Claritin D12 and D24 commercial products are combinations of loratadine and pseudoephedrine and the “Allegra-D” commercial product is a combination of fexofenadine and pseudoephedrine. See, Physicians Desk Reference 2000.

However, the administration of pseudoephedrine may cause unwanted side effects. Side effects associated with pseudoephedrine include insomnia, dizziness, weakness, tremor or arrhythmia. These and other unwanted side effects may cause patients suffering from congestion associated with allergic disorders to avoid or discontinue treatment with pseudoephedrine-containing products.

There is a need for a clinically effective therapy to treat or prevent such severe nasal blockage and/or congestion associated with allergic and inflammatory conditions of the airway passages in a human with a non-sedating antihistamine which does not provide the potential adverse side effects that may be experienced with products containing pseudoephedrine. That is, there is a need for a non-sedating antihistamine that provides decongestant effect to reduce the amount of, or eliminate the need for, an additional decongestant such as pseudoephedrine.

### SUMMARY OF THE INVENTION

The present invention provides a method for treating and/or preventing nasal congestion associated with allergic and inflammatory conditions of the airway passages in a human in need thereof comprising administering to said human an amount of desloratadine effective for such treating and/or preventing.

There is also disclosed a method of treating and/or preventing nasal blockage associated with allergic and inflammatory conditions of the airway passages in a human in need thereof comprising administering to said human an amount of desloratadine effective for such treating and/or preventing.

There is also disclosed a method of treating and/or preventing severe nasal blockage associated with allergic and inflammation conditions in a human in need thereof comprising administering to said human an amount of desloratadine effective for such treating and/or preventing in combination with one or more additional decongestants.

There is also disclosed a method for treating and/or preventing nasal congestion associated with allergic and inflammatory conditions of the airway passages in a human in need thereof comprising administering to said human an amount of antihistamine effective for such treating and/or preventing, wherein the antihistamine is selected from the group consisting of loratadine, cetirizine, fexofenadine, ebastine, astemizole, norastemizole, epinastine, efletirizine or a pharmaceutically acceptable salt thereof.

There is also disclosed method of treating and/or preventing severe nasal blockage associated with allergic and inflammation conditions in a human in need thereof comprising administering to said human an amount of antihistamine effective for such treating and/or preventing in combination with one or more additional decongestants, wherein the antihistamine is selected from the group consisting of loratadine, cetirizine, fexofenadine, ebastine, astemizole, norastemizole, epinastine, efletirizine or a pharmaceutically acceptable salt thereof.

### DETAILED DESCRIPTION OF INVENTION

The phrase “allergic and inflammatory conditions of the airway passages” means those allergic and inflammatory conditions and symptoms found and in the upper and lower airway passages from the nose to the lungs. Typical allergic and inflammatory conditions or upper and lower airway passages include seasonal and perennial allergic rhinitis, non-allergic rhinitis, asthma including allergic and non-allergic asthma, sinusitis, and colds.

The term “congestion” means an obstruction with a decreased diameter of the nasal cavity and an increased nasal airflow resistance, thus decreasing the obstruction, stuffiness or blockage of the upper airway passages and/or the constriction of the lower airway passages from the nose to the lungs, including severe nasal blockage and congestion.

The term “nasal blockage” means a subject suffering from congestion such that the subject has complaints of moderate to severe nasal obstruction and blockage of nasal airflow, typically with less rhinorrhea. For example, the subjects should demonstrate at the baseline visit, the 7 twice daily (in the AM and in the PM) run-in diary reflective scores that includes the three calendar days prior to baseline, as well as the AM of the baseline day. The scores may be as follows: the total rhinorrhea score should equal at least 14; the total nasal congestion score should exceed the total rhinorrhea score by at least two; the total nasal symptom score should equal at least 42; the total non-nasal symptom score should equal at least 28.

The primary efficacy variable is the average AM-PM “reflective” nasal congestion symptom score, expressed as change from Baseline averaged over the treatment period (Days 1 through 8). The primary contrast for this variable will be for example, desloratadine versus placebo.

Secondary efficacy parameters include the AM “instantaneous” nasal congestion score, as well as the average AM-PM “reflective” and “instantaneous” total symptom score minus nasal congestion (sum of six individual symptom scores); total symptom score (sum of the seven individual symptom scores including nasal congestion); nasal symptom score (sum of the four nasal symptom scores); total nasal symptom score minus nasal blockage/airflow; non-nasal symptom score (sum of the three individual non-nasal symptom scores); each of the seven individual symptom scores and subject and investigator/designee evaluation of therapeutic response to treatment.

In addition, and on an exploratory basis, an objective measure of freedom of nasal breathing/degree of nasal blockage will be carried out twice daily by the subject, using an In-Check Nasal Inspiratory Flow Meter device, and the findings recorded on subject diaries. Any correlation with the subjective symptom of nasal congestion will also be evaluated.

Assessment of safety will include routine subject- and investigator-evaluated adverse events; investigator-evaluated vital signs and ECG; laboratory evaluations of blood chemistry, hematology, and urinalysis.

Subjects can be of either sex and any race at least 12 years of age; at least a 2 year history of moderate/severe spring seasonal allergic rhinitis, documented by positive skin prick test reactivity to the prevalent spring seasonal allergen(s) to which the patient is sensitive (history of allergy/skin test responsiveness to perennial allergens and molds will also be obtained for demographic purposes and for possible ad hoc sub-group analyses.

As set forth above, in order to qualify at screening for this study, the subject must demonstrate the following reflective (prior 12 hours) signs/symptoms scores: a nasal rhinorrhea (anterior or posterior) score of at least moderate (score of at least 2), nasal congestion must be at least moderate (score of at least 2), total nasal symptom score must total at least 6, and total non-nasal score must equal at least 4. In order to qualify for randomization at the Baseline visit, the seven twice-daily (AM and PM) run-in diary “reflective” scores, which includes the three calendar days prior to Baseline, as well as the AM of the Baseline day, must be as follows: the symptom score for rhinorrhea must equal at least 14; the total nasal congestion score must exceed the total rhinorrhea score by at least 2 points; the total nasal symptom score must equal at least 42; and the total non-nasal symptom score must equal at least 28.

Subjects must be in good health, free of any clinically significant disease, other than SAR, that might interfere with the study schedule, evaluation of SAR, or interpretation of study-derived data. Rescue medication will not be provided. Group will receive desloratadine 5.0 mg; Group II will receive placebo. Placebo tablet for desloratadine 5.0 mg. The study will be double blind, parallel groups, and carried out at multiple centers.

This is a pilot, probing study. As such, the sample size was selected to provide reasonable estimates and trends for a possible treatment effect. Therefore, with a sample size of 150 subjects per treatment group, a two-tailed alpha level of 0.05, and a pooled standard deviation of 0.6 points on the change from Baseline, a difference of approximately 0.19 units or more between treatment groups can be detected with a power of at least 80%.

300 patients will be enrolled at approximately 10 sites, approximately 30 subjects enrolled per site. The Ratio of subjects assigned to treatments will be 1:1, desloratadine to placebo, respectively.

Subjects will be randomized to the two treatment randomization schedule.

Randomization will be performed in appropriately sized blocks using random numbers generated by SAS function UNIFORM with seed based on clock-time. Drop-outs will not be replaced at sites.

This is a one-week, pilot Phase II study, composed of a total of 300 subjects to be enrolled. Each subject will be screened during the spring allergy season in the United States. Subjects must be sufficiently symptomatic with respect to nasal and non-nasal symptoms at both the Screening and Baseline visits. Subjects in the two treatment groups will be required to visit the office for a Baseline visit, usually 3 to 14 days after the screening visit, in order to qualify to receive the first dose of randomized treatment unit. In each treatment group, subjects will be required to re-visit the office for one follow-up visit, on Day 8 (7 days after Baseline).

The amount of desloratadine effective for treating or preventing congestion associated with allergic and inflammatory conditions of the airway passages will vary with the age, sex, body weight and severity of the allergic and inflammatory condition of the patient. Typically, the amount of desloratadine effective for treating or preventing such allergic and inflammatory conditions is in the range of about 2.5 mg/day to about 45 mg/day, preferably about 2.5 mg/day to about 20 mg/day, or about 5.0 mg/day to about 15 mg/day, or about 5.0 mg/day to about 10 mg/day, more preferably about 5.0 mg/day to about 7.5 mg/day, and most preferably about 5.0 mg/day in single or divided doses, or a single dose of 5.0 mg/day.

U.S. Pat. No. 4,659,716 discloses methods of making desloratadine, pharmaceutical compositions containing it and methods of using desloratadine and pharmaceutical compositions containing it to treat allergic reaction in mammals. U.S. Pat. No. 5,595,997 discloses pharmaceutical compositions containing desloratadine and methods of using desloratadine for treating and preventing various disease states, e.g., allergic rhinitis. Desloratadine is available from Schering Corporation, Kenilworth, N.J.

The antihistamines that are also useful in the present invention include loratadine, cetirizine, levocetirizine, mizolastine for seasonal allergic rhinitis, fexofenadine, ebastine, astemizole, norastemizole, epinastine, efletirizine or a pharmaceutically acceptable salt thereof. The use of desloratadine, loratadine, fexofenadine and cetirizine is most preferred.

Cetirizine reportedly is disclosed in U.S. Pat. No. 4,525,358. Preferably the pharmaceutically acceptable salt is the hydrochloride, also known as cetirizine hydrochloride. The amount of cetirizine which can be employed in a unit dosage form of the present composition can range from about 2.5 to 20 mg, also from about 5 to about 10 milligrams, preferably about 10 milligrams.

Fexofenadine (MDL 16,455A) is reportedly disclosed in U.S. Pat. No. 4,254,129. Preferably the pharmaceutically acceptable salt is the hydrochloride, also known as fexofenadine hydrochloride. The amount of fexofenadine which can be employed in a unit dosage form of the present composition can range from about 40 to 200 mg, also from about 60 to about 180 milligrams, also about 120 milligrams.

Ebastine reportedly is described in EP 134124. The amount of ebastine which can be employed in a unit dosage form can range from about 5 to about 20 mg, preferably about 10 mg.

Astemizole reportedly is described in U.S. Pat. No. 4,219,559. The amount of astemizole which can be employed in a unit dosage form can range from about 5 to about 20 mg, preferably about 10 mg.

Norastemizole reportedly is an antihistamine, whose technical name is 1-((4-fluorophenyl)methyl)-N-4-piperidinyl-1H-benzimidazol-2-amine. The compound is an active metabolite of astemizole. The amount of norastemizole which can be employed in a unit dosage form can range from about 5 to about 40 mg, also from about 10 to about 20 mg.

Epinastine reportedly is described in DE 3008944 or Jpn. J. Clin. Pharmocol. Ther., 1991, 22, page 617. The amount of epinastine which can be employed in a unit dosage form can range from about 1 to about 20 mg, preferably about 2 to about 18 mg.

Efletirizine (UCB-28754) reportedly is an antihistamine, whose technical name is [2-[4-[Bis(p-fluorophenyl)methyl]-1-piperazinyl]ethoxy]acetic acid. CASReg. No. 140756-35-7. The amount of efletirizine which can be employed in a unit dosage form can range from about 4 to about 60 mg.

U.S. Pat. No. 4,282,233 discloses methods of making loratadine, pharmaceutical compositions containing it and methods of using loratadine and pharmaceutical compositions containing it to effect an anti-allergic response in mammals. Loratadine is available from Schering-Plough Corporation, Kenilworth, N.J. under the Claritin™ Tradename.

The pharmaceutical compositions of desloratadine and/or other antihistamines can be adapted for any mode of administration e.g., for oral, parenteral, e.g., subcutaneous (“SC”), intramuscular (“IM”), intravenous (“IV)” and intraperitoneal (“IP”), topical or vaginal administration or by inhalation (orally or intranasally). Preferably desloratadine and/or other antihistamines is administered orally.

Such pharmaceutical compositions may be formulated by combining desloratadine and/or other antihistamines or an equivalent amount of a pharmaceutically acceptable salt thereof with a suitable, inert, pharmaceutically acceptable carrier or diluent that may be either solid or liquid. Desloratadine may be converted into the pharmaceutically acceptable acid addition salts by admixing it with an equivalent amount of a pharmaceutically acceptable acid. Typically suitable pharmaceutically acceptable acids include the mineral acids, e.g., HNO₃, H₂SO₄, H₃PO₄, HCl, HBr, organic acids, including, but not limited to, acetic, trifluoroacetic, propionic, lactic, maleic, succinic, tartaric, glucuronic and citric acids as well as alkyl or arylsulfonic acids, such as p-toluenesulfonic acid, 2-naphthalenesulfonic acid, or methanesulfonic acid. The preferred pharmaceutically acceptable salts are trifluoroacetate, tosylate, mesylate, and chloride. Desloratadine is more stable as the free base than as an acid addition salt and the use of the desloratadine free base in pharmaceutical compositions of the present invention is more preferred.

Solid form preparations include powders, tablets, dispersible granules, capsules, cachets and suppositories. The powders and tablets may be comprised of from about 5 to about 95 percent active ingredient. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. As an example may be mentioned water or water-propylene glycol solutions for parenteral injection. Solid form preparations may be converted into liquid preparations shortly before use for either oral or administration. Parenteral forms to be injected intravenously, intramuscularly or subcutaneously are usually in the form of sterile solutions and may contain tonicity agents (salts or glucose), and buffers. Opacifiers may be included in oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g., nitrogen.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

In addition, the present invention includes combinations of desloratadine and/or other antihistamines and other decongestants. Due to the decongestant effect of desloratadine and/or other antihistamines, the other decongestants may be present in a reduced amount compared to other combinations of antihistamines and decongestants. Other decongestants which may be used in combination with desloratadine and/or other antihistamines include pseudoephedrine, phenylephrine and phenylpropanolamine.

Preferably, the pharmaceutical preparation is in a unit dosage form. In such form, the preparation is subdivided into suitably sized unit doses containing appropriate quantities of the active component, e.g., an effective amount to achieve the desired purpose.

Desloratadine is particularly useful for the treatment and prevention of the nasal (stuffiness/congestion, rhinorrhea, nasal itching, sneezing) and non-nasal (itchy/burning eyes, tearing/watery eyes, redness of the eyes, itching of the ears/palate) symptoms of seasonal allergic rhinitis, including nasal congestion, in patients in need of such treating and/or preventing.

The clinical efficacy and safety of desloratadine has been documented in over 3,200 seasonal allergic rhinitis patients in four double-blinded, randomized clinical trials. The results of these clinical studies demonstrated the efficacy of desloratadine in the treatment of adult and adolescent patients with seasonal rhinitis.

Efficacy endpoints in all the studies were Total Symptom Score, Total Nasal Symptom Score, Total Non-nasal Symptom Score, and Health Quality of Life (HQOL) analysis in efficacy trials. Desloratadine (5 mg once daily) significantly reduced the total symptom scores (the sum of individual scores for rhinorrhea, sneezing, congestion/stuffiness, nasal itching, itchy/burning eyes, tearing, ocular redness, and itchy ears/palate). Desloratadine (5 mg) was significantly (p<0.01) more effective than placebo in reducing nasal symptoms. An important efficacy endpoint analyzed in the desloratadine studies is the AM NOW total symptom score. This parameter measures the total symptom relief by the patient after 24 hours before taking the next day dose. Statistically significant (p<0.05) reductions were maintained for the full 24 hour dosing interval over the entire 5 mg to 20 mg dosage range.

The effects of desloratadine on nasal congestion/stuffiness are described by using data pooled from randomized, parallel-group, double-blind, placebo-controlled studies of desloratadine in patients with SAR. Patients (12-75 years; pooled n-659-662/group) with a ≧2-year history of seasonal allergic rhinitis and moderate-to-10 severe symptoms present at the time of enrollment received desloratadine (5 mg or 7.5 mg) or placebo PO once a day for 14 days. The severity (0=none, 1=mild, 2=moderate, 3=severe) of congestion/stuffiness was assessed by patients for the study duration. The 14-day average change in symptom severity score from baseline was assessed. The mean symptom severity score for nasal congestion/stuffiness was 2.4 in each treatment group at baseline, indicating patients had moderate-to-severe nasal congestion before receiving treatment. Desloratadine significantly decreased nasal congestion/stuffiness (P=0.02 and 0.01 for 5 mg and 7.5 mg respectively, of desloratadine vs placebo) as well as total symptom severity.

These data indicate that desloratadine has the added benefit of providing significant relief from persistent allergic symptoms such as nasal congestion/stuffiness in patients with SAR.

1-5. (canceled)

6. A method of treating and/or preventing nasal blockage associated with allergic and inflammatory conditions of the airway passages in a human in need thereof comprising administering desloratadine effective for such treating and/or preventing.

7. The method of claim 6, wherein the amount of desloratadine is in the range of about 2.5 mg/day to about 45 mg/day.

8. The method of claim 7, wherein the amount of desloratadine is about 5 mg/day to about 15 mg/day.

9. The method of claim 8, wherein the amount of desloratadine is about 5 mg/day to about 10 mg/day.

10-41. (canceled)

42. The method of claim 9, wherein the amount of desloratadine is about 5 or about 6 mg/day.

43. The method of any of claims 6 to 9, and 42 further comprising one or more additional decongestants.

44. The method of claim 43, wherein said decongestant is pseudoephedrine.

45. The method of claim 44, wherein said pseudoephedrine is present in an amount to provide decongestant effect with elimination or reduction of an adverse side effect associated therewith.

46. The method of claim 45, wherein said adverse side effect is insomnia, dizziness, weakness, tremor, or arrhythmia.
